(19) United States
(12) Patent Application Publication
(10) Pub. No.: US 2026/0117299 A1
(43) Pub. Date: Apr. 30, 2026

(54) SPATIAL ANALYSIS USING PORE SEQUENCERS

(71) Applicant: 10x Genomics, Inc., Pleasanton, CA (US)

(72) Inventors: Hanyoup Kim, Foster City, CA (US); Zixue Ma, Pleasanton, CA (US); Nabil Mikhaiel, Pleasanton, CA (US); Carolyn A. Morrison, Pleasanton, CA (US)

(21) Appl. No.: 19/364,513

(22) Filed: Oct. 21, 2025

Related U.S. Application Data
(60) Provisional application No. 63/714,549, filed on Oct. 31, 2024.
(60) Provisional application No. 63/809,803, filed on May 21, 2025.

(51) Int. Cl. C12Q 1/6874; G01N 33/68

(52) U.S. Cl. CPC C12Q 1/6874; G01N 33/6818

(57) ABSTRACT

Provided herein are methods of interrogating spatial gene expression in a sample using substrates having sequencing pores. The disclosed methods allow for spatial analysis of analytes from biological samples using a pore-based sequencing approach and without the need for a barcoded spatial array. In some embodiments, an analyte or intermediate agent thereof, is released from a biological sample and directly sequenced by traversing through pores of a sequencing array including a plurality of pores, e.g., nanopores. In some embodiments, light or other stimuli is used to release an analyte or intermediate agent thereof from a specific region of interest in the biological sample followed by sequencing using sequencing array including a plurality of pores.

### CROSS-REFERENCE TO RELATED APPLICATIONS

This application the benefit to U.S. Provisional Ser. No. 63/714,549 , filed Oct. 31, 2024; and U.S. Provisional Ser. No. 63/809,803 , filed May 21, 2025. The contents of each of these applications are incorporated herein by reference in their entireties.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Generally, targeting a particular analyte in a biological sample utilizes a capture probe that targets a common transcript sequence such as a poly(A) mRNA-like tail. However, this approach is capable of detecting a high number of off target analytes. Methods such as RNA-templated ligation offer an alternative to indiscriminate capture of a common transcript sequence. See, e.g., Yeakley, PLoS One, 25; 12(5):e0178302 (2017), which is incorporated by reference in its entirety. However, there remains a need to develop an alternative to common transcript sequence (e.g., poly(A) mRNA-like tail) capture of target analytes that is capable of detecting an analyte(s) in an entire transcriptome while providing information regarding the spatial location and abundance of a target analyte.

Most spatial analysis requires off-substrate sequencing steps of in situ detection using target specific probes. There remains a need to develop additional methods for spatial analysis

### SUMMARY

In the present disclosure, the Applicant has identified that spatial analysis can be performed using a substrate having a plurality of pores, which are used for sequencing. The methods described herein provide a fast and simple workflow without the need for spatial barcodes or capture probes on an array. In addition, because the methods are performed on a substrate, no complicated and time consuming library preparation steps are required. Finally, the methods allow for alternative methods of spatial resolution, including light-based release mechanisms that allow for entire or partial tissue spatial analysis.

Thus, included herein is a method for determining location of an analyte or an intermediate agent in a biological sample. In some instances, the method includes (a) aligning a first substrate comprising the biological sample with a sequencing array on a second substrate, wherein the sequencing array comprises a plurality of pores, such that at least a portion of the biological sample is aligned with at least a portion of the sequencing array on the second substrate; (b) migrating the analyte or the intermediate agent from the biological sample to the sequencing array; and (c) passing the analyte or the intermediate agent through a pore of the sequencing array, thereby sequencing the analyte or the intermediate agent, and using the sequence of the analyte or the intermediate agent and the location of the pore through which the analyte or the intermediate agent passed to determine the location of the analyte or the intermediate agent in the biological sample. In some instances, the method include steps (b), (c), and (d).

Also disclosed herein is a method for determining location of an analyte or an intermediate agent in a region of interest in a biological sample, wherein the method includes (a) providing a sequencing array on a second substrate, wherein the sequencing array comprises a plurality of pores, optionally aligning a first substrate comprising the biological sample with the sequencing array such that at least a portion of the biological sample is aligned with at least a portion of the sequencing array on the second substrate; (b) releasing the analyte or the intermediate agent from the region of interest in the biological sample; (c) migrating the analyte or the intermediate agent from the region of interest in the biological sample to a pore of the sequencing array; and (d) passing the analyte or the intermediate agent through the pore of the sequencing array, thereby sequencing the analyte or the intermediate agent, and using the sequence of the analyte or the intermediate agent and the location of the region of interest in the biological sample to determine the expression of the analyte or the intermediate agent in the region of interest in the biological sample.

In some instances, light is used to release the analyte or the intermediate agent from the region of interest in the biological sample. In some instances, the light is ultraviolet light. In some instances, the releasing step comprises exposing at least two regions of interest in the biological sample to light, optionally wherein the light exposed to at least two regions of interest is ultraviolet light. In some instances, the releasing step comprises exposing the multiple regions of interest in the biological sample at the same time, optionally wherein analytes or intermediate agents from each region of interest in the multiple regions of interest do not laterally diffuse to other regions of interest. In some instances, the methods also include, after step (d): selecting a second region of interest in the biological sample; and repeating steps (b) through (d) to determine a location of a second analyte or a second intermediate agent in the second region of interest in the biological sample.

In some instances, the methods include determining the location of the analyte or the intermediate agent in the biological sample at a first time point followed by determining the location of the second analyte or the second intermediate agent in the second region of interest in the biological sample at a second time point, wherein the second time point is after the first time point.

In some instances, locations of the analyte and the second analyte are determined based on timing in which the analyte, the intermediate agent, the second analyte, or the second intermediate agent pass through the pore of the sequencing array. In some instances, the methods include, prior to step (b), applying a mask to the second substrate in a location not aligned with the region of interest.

Also disclosed is a method for determining location of an analyte or an intermediate agent in a region of interest in a biological sample, wherein the method includes (a) providing a sequencing array on a second substrate, wherein the sequencing array comprises a plurality of pores, optionally aligning a first substrate comprising the biological sample with the sequencing array such that at least a portion of the biological sample is aligned with at least a portion of the sequencing array on the second substrate; (b) applying a mask to the first substrate in a location not aligned with the region of interest; (c) releasing the analyte or the intermediate agent from the region of interest in the biological sample; (d) migrating the analyte or the intermediate agent from the region of interest in the biological sample to a pore of the sequencing array; and (e) passing the analyte or the intermediate agent through the pore of the sequencing array, thereby sequencing the analyte or the intermediate agent, and using the sequence of the analyte or the intermediate agent and the location of the region of interest in the biological sample to determine the expression of the analyte or the intermediate agent in the region of interest in the biological sample.

In some instances, light is used to release the analyte or the intermediate agent from the region of interest in the biological sample. In some instances, the light is ultraviolet light. In some instances, the light is infrared light. In some instances, the releasing step comprises exposing at least two regions of interest in the biological sample to light, optionally wherein the light exposed to at least two regions of interest is ultraviolet light or infrared light. In some instances, the releasing step comprises exposing the multiple regions of interest in the biological sample at the same time, optionally wherein analytes or intermediate agents from each region of interest in the multiple regions of interest do not laterally diffuse to other regions of interest. In some instances, the methods include, after step (d): selecting a second region of interest in the biological sample; and repeating steps (b) through (d) to determine a location of a second analyte or a second intermediate agent in the second region of interest in the biological sample. In some instances, the methods include determining the location of the analyte or the intermediate agent in the biological sample at a first time point followed by determining the location of the second analyte or the second intermediate agent in the second region of interest in the biological sample at a second time point, wherein the second time point is after the first time point. In some instances, locations of the analyte and the second analyte are determined based on timing in which the analyte, the intermediate agent, the second analyte, or the second intermediate agent pass through the pore of the sequencing array.

Also disclosed is a method for assaying an analyte in a region of interest in a biological sample, wherein the method includes: (a) providing a sequencing array on a second substrate, wherein the sequencing array comprises a plurality of pores, aligning a first substrate comprising the biological sample with the sequencing array such that at least a portion of the biological sample is aligned with at least a portion of the sequencing array on the second substrate; (b) releasing the analyte, or an intermediate agent thereof, from the region of interest in the biological sample on the first substrate; (c) migrating the analyte or the intermediate agent thereof from the region of interest in the biological sample on the first substrate to the sequencing array on the second substrate; and (d) traversing the analyte or the intermediate agent thereof through a pore of the sequencing array to sequence the analyte or the intermediate agent thereof, and using the sequence of the analyte or the intermediate agent thereof and the location of the region of interest in the biological sample to determine the presence of the analyte in the region of interest in the biological sample.

In some embodiments, infrared light is used to release the analyte or intermediate agent from the region of interest in the biological sample. In some embodiments, the biological sample is exposed to or contacted with the infrared light. In some embodiments, exposure to infrared light locally increases the temperature of the biological sample in the region of interest, locally denatures polynucleotides (e.g., the analyte or intermediate agent thereof) present in the region of interest, and/or locally increases enzymatic activity in the region of interest.

In some instances, the first substrate comprises a conductive layer. In some instances, the second substrate comprises a conductive layer. In some instances, a flow cell is placed between the first substrate and the second substrate. In some instances, one or more adhesives connect the first substrate and the second substrate to the flow cell. In some instances, the sequencing array comprises partitions around each pore.

In some instances, the methods include washing the first substrate and/or the second substrate.

In some instances, the sequencing array comprises a pillar region above each pore, thereby reducing crowding of analytes or intermediate agents inside of the pores compared to a sequencing array without the pillar region. In some instances, the migrating comprises applying an electric field to first substrate and the second substrate. In some instances, the analyte or intermediate agent is a nucleic acid, wherein each type of nucleotide in the analyte or the intermediate agent has an electronic signature when passing through the pore of the sequencing array which is distinguishable from the electronic signature of each other type of nucleotide in the analyte or the intermediate agent. In some instances, release of the analyte or intermediate agent from the biological sample is controlled by varying concentration of a releasing enzyme. In some instances, the releasing enzyme is an RNase enzyme. In some instances, release of the analyte or intermediate agent from the biological sample is controlled by varying temperature of the first substrate. In some instances, release of the analyte or intermediate agent from the biological sample is controlled by varying light intensity, illumination size, and/or wavelength. In some instances, the sequencing array comprises at least 10, 100, 1000, or 10,000 pores.

In some instances, the methods include mounting a biological sample on the first substrate. In some instances, the biological sample is embedded in a hydrogel. In some instances, the biological sample is removed from the hydrogel, and the analyte or the intermediate agent remains in the hydrogel. In some instances, the biological sample is removed from the first substrate. In some instances, the analyte or the intermediate agent remains in a hydrogel on the first substrate.

In some instances, the methods include imaging the biological sample. In some instances, the methods include staining the biological sample, wherein the staining comprises immunofluorescence, immunohistochemistry, hematoxylin, and/or eosin staining.

In some instances, the methods include treating the biological sample with a permeabilization agent. In some instances, the permeabilization agent comprises a protease selected from trypsin, pepsin, elastase, or proteinase K. In some instances, the analyte or intermediate agent is a nucleic acid, wherein the analyte or intermediate agent comprises a mutation, optionally wherein the mutation comprises an insertion, a deletion, a substitution, or a splicing error.

In some instances, the analyte is RNA. In some instances, the analyte is mRNA. In some instances, the analyte or intermediate agent is DNA. In some instances, the analyte or intermediate agent is genomic DNA or cDNA. In some instances, the intermediate agent comprises amplified DNA. In some instances, the intermediate agent comprises cDNA.

In some instances, the methods disclosed herein also include hybridizing a first probe and a second probe to the analyte in the biological sample, wherein the analyte is a nucleic acid, wherein the first probe and the second probe each comprise a sequence that is substantially complementary to sequences of the analyte; and coupling the first probe and the second probe, thereby generating a connected probe hybridized to the analyte in the biological sample. In some instances, the methods also include releasing the connected probe from the analyte, optionally wherein the releasing comprises contacting the biological sample with a reagent medium. In some instances, the reagent medium comprises an agent for releasing the connected probe, thereby permeabilizing the biological sample and releasing the connected probe from the analyte. In some instances, the agent for releasing the connected probe comprises an RNase, wherein the RNase is selected from RNase A, RNase C, RNase H, or RNase I.

In some instances, the first probe and the second probe hybridize to adjacent sequences of the analyte. In some instances, the first probe and the second probe hybridize to sequences in the analyte that are at least 1, 2, 3, 4, 5, 6, 7, 8, 9, 10, or more nucleotides apart. In some instances, the coupling the first probe and the second probe comprises ligating the first probe and the second probe.

In some instances, after the hybridizing, the methods include generating: (i) an extended first probe or (ii) an extended second probe by contacting with a polymerase, wherein the extended first probe comprises a sequence complementary to a sequence between the sequence hybridized to the first probe and the sequence hybridized to the second probe, and the extended second probe comprises a sequence complementary to a sequence between the sequence hybridized to the first probe and the sequence hybridized to the second probe. In some instances, the coupling the first probe and the second probe comprises ligating: (i) the first probe and the extended second probe, (ii) the extended first probe and the second probe, or (iii) the extended first probe and the extended second probe. In some instances, the ligating is performed using a ligase. In some instances, ligase is a Chlorella virus DNA ligase, a single-stranded DNA ligase, or a T4 DNA ligase. In some instances, the first probe and the second probe are comprised in a plurality of probes that target about 100, about 500, about 1000, about 2000, or about 5000 analytes.

In some instances, the analyte is a protein. In some instances, the methods include contacting the biological sample with a plurality of analyte capture agents, wherein an analyte capture agent of the plurality of analyte capture agents comprises an analyte binding moiety and a capture agent barcode domain, wherein the capture agent barcode domain comprises an analyte binding moiety barcode that uniquely identifies the analyte binding moiety and/or the protein, and wherein upon the contacting, the analyte binding moiety binds to the protein in the biological sample. In some instances, the analyte binding moiety is an antibody or a functional fragment thereof. In some instances, the capture agent barcode domain is coupled to the analyte binding moiety by a linker. In some instances, the linker is a cleavable linker. In some instances, the cleavable linker is a disulfide linker, a photo-cleavable linker, a UV-cleavable linker, or an enzyme cleavable linker. In some instances, the analyte capture agent is comprised in a plurality of analyte capture agents that target about 100, about 500, about 1000, about 2000, or about 5000 analytes.

In some instances, the biological sample is a tissue sample, preferably a tissue section. In some instances, the biological sample is a fresh-frozen tissue section. In some instances, the biological sample is a fixed tissue section. In some instances, the biological sample is a formalin-fixed paraffin-embedded tissue section.

Also disclosed is a system. In some instances, the system includes a first substrate comprising a biological sample; and a second substrate comprising a sequencing array comprising a plurality of pores. In some instances, the system includes a flow cell between the first substrate and the second substrate. In some instances, the system includes a light source, optionally wherein the light source is for releasing an analyte from the biological sample. In some instances, the system includes a mask to inhibit release of analytes in regions that are not of interest in the biological sample. In some instances, the system further includes a reverse transcriptase. In some instances, the system further includes a polymerase. In some instances, the polymerase is a DNA polymerase. In some instances, the system includes a plurality of dNTPs. In some instances, the system includes one or more permeabilization reagents.

In some instances, the one or more permeabilization reagents includes a protease, a surfactant, or a detergent. In some instances, the protease includes proteinase K, pepsin, or collagenase. In some instances, the system further includes an RNase. In some instances, the system further includes a DNase. In some instances, the system further includes a first probe and a second probe, wherein each of the first probe and the second probe comprises a sequence that is substantially complementary to sequences of the analyte. In some instances, the system further includes a ligase. In some instances, the ligase is a Chlorella virus DNA ligase, a single-stranded DNA ligase, or a T4 DNA ligase.

Also disclosed herein is a kit. In some instances, the kit includes a first substrate comprising a biological sample; a second substrate comprising a sequencing array comprising a plurality of pores; and instructions for performing any one of the methods disclosed herein.

In some instances, the kit includes a flow cell between the first substrate and the second substrate. In some instances, the kit includes a light source to release an analyte from the biological sample. In some instances, the kit includes a mask to inhibit release of analytes in regions that are not of interest in the biological sample. In some instances, the kit includes a reverse transcriptase. In some instances, the kit includes a polymerase. In some instances, the polymerase is a DNA polymerase. In some instances, the kit includes a plurality of dNTPs. In some instances, the kit includes one or more permeabilization reagents. In some instances, the one or more permeabilization reagents comprises a protease, a surfactant, or a detergent. In some instances, the kit includes protease comprises proteinase K, pepsin, or collagenase. In some instances, the kit includes an RNase. In some instances, the kit includes a DNase. In some instances, the kit includes a first probe and a second probe, wherein each of the first probe and the second probe comprises a sequence that is substantially complementary to sequences of a nucleic acid analyte. In some instances, the kit includes a ligase. In some instances, the ligase is a Chlorella virus DNA ligase, a single-stranded DNA ligase, or a T4 DNA ligase.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe and an analyte capture agent.

FIG. 12 shows a flowchart for a spatial transcriptomics embodiment disclosed herein.

FIG. 13 shows a flowchart for a spatial transcriptomics embodiment disclosed herein.

FIG. 14 shows a schematic of two substrates, one of which includes a plurality of pores through which analytes or intermediate agents pass to be sequenced.

FIG. 15A shows a schematic in which a single light source releases analytes or intermediate agents from a region of interest in a biological sample.

FIG. 15B shows a schematic in which multiple light sources release analytes or intermediate agents from a region of interest in a biological sample.

FIG. 16 shows a flowchart for a spatial transcriptomics embodiment disclosed herein.

FIG. 17 shows a schematic of two substrates, one of which includes a plurality of pores through which analytes or intermediate agents migrate and are sequenced.

### I. DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457 , 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434): 1463-1467, 2019; Lee et al., Nat. Protoc. 10(3): 442-458, 2015; Trejo et al., PLoS ONE 14(2): e0212031, 2019; Chen et al., Science 348(6233): aaa6090, 2015; Gao et al., BMC Biol. 15: 50, 2017; and Gupta et al., Nature Biotechnol. 36: 1197-1202, 2018; and the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), both of which are available at the 10x Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples - which can be from different tissues or organisms - assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 October 1; 9(10): 5188-96; Kap M. et al., PLoS One.; 6(11): e27704 (2011); and Mathieson W. et al., Am J Clin Pathol.; 146(1): 25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I) (d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588: 63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

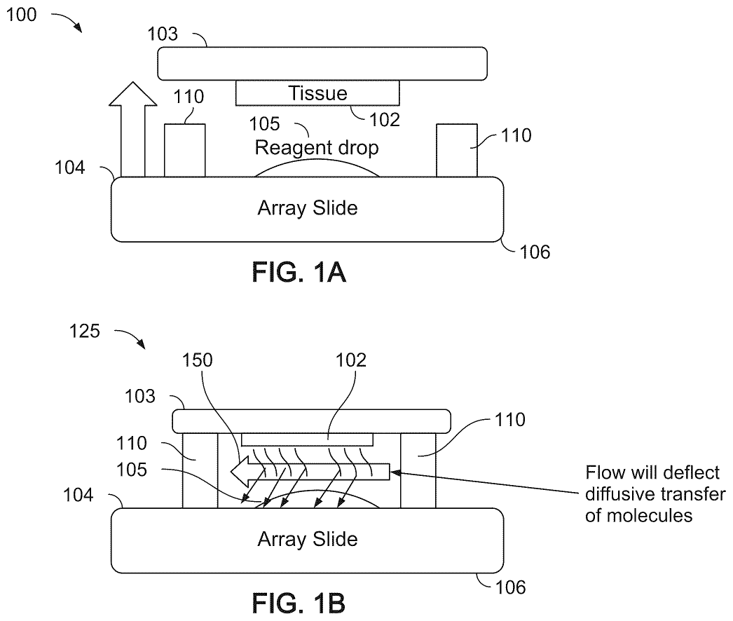

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

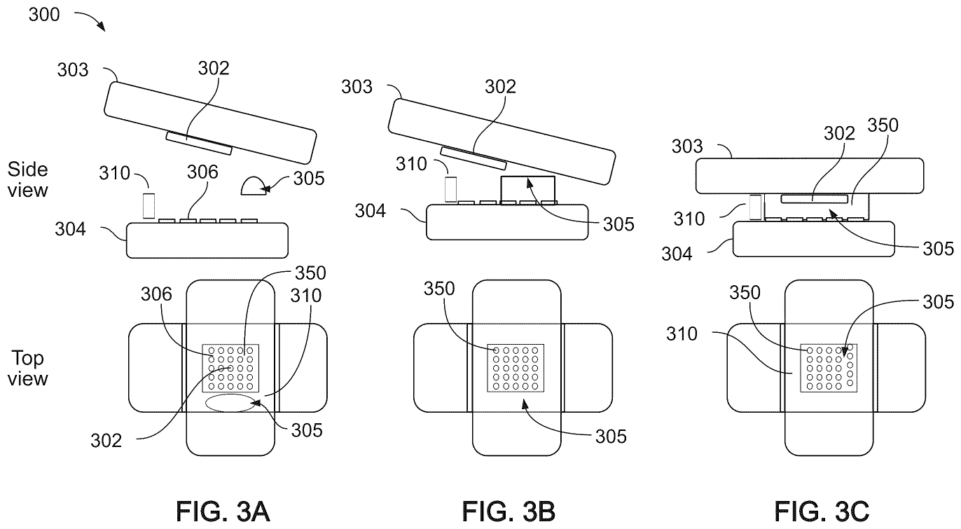

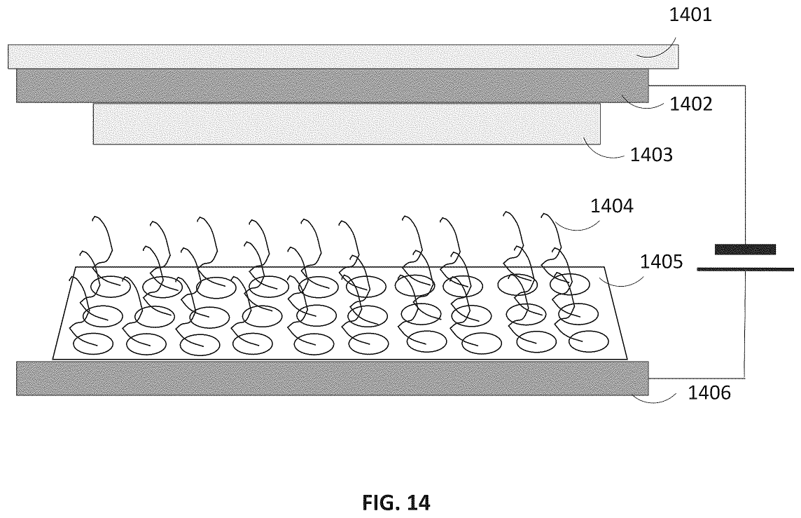

During the exemplary sandwiching process, the first substrate is aligned with the second substrate, such that at least a portion of the biological sample is aligned with at least a portion of the capture probes (e.g., aligned in a sandwich configuration). “Aligning” as used herein refers to arranging two substrates (e.g., a slide 103 with an array slide 104 as shown in FIGS. 1A and 1B or a slide 303 and an array slide 304 as shown in FIGS. 3A-3C) proximal to each other. In one embodiment, as aligned, the plane of the slide having the biological sample and the plane of the slide having sequencing pores face one another. This is illustrated, for example, in FIG. 14, which shows a biological sample 1403 on a substrate 1401 that faces a second substrate 1406 having sequencing pores.

As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

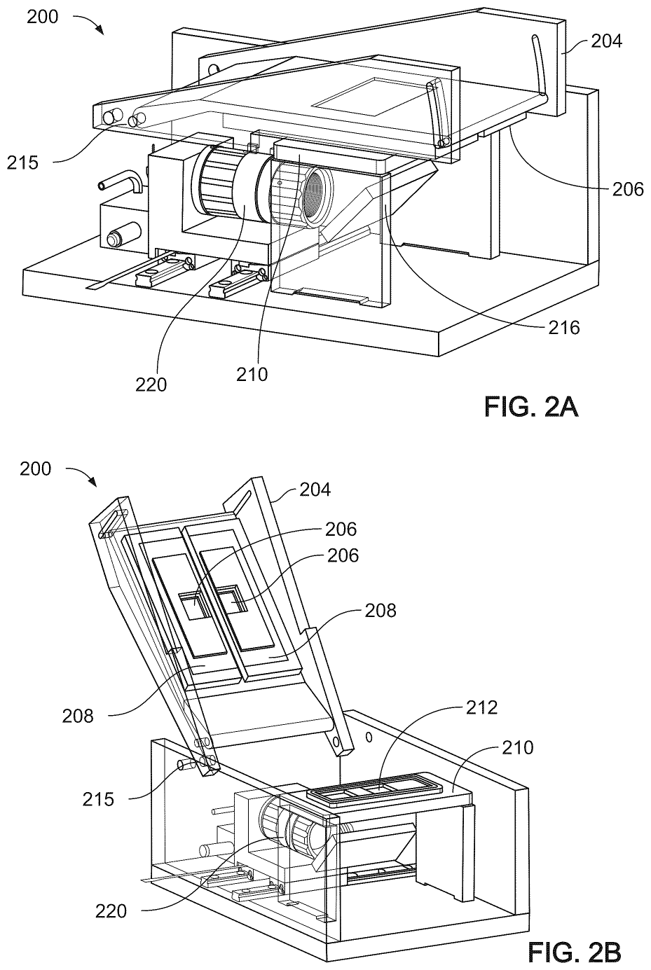

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

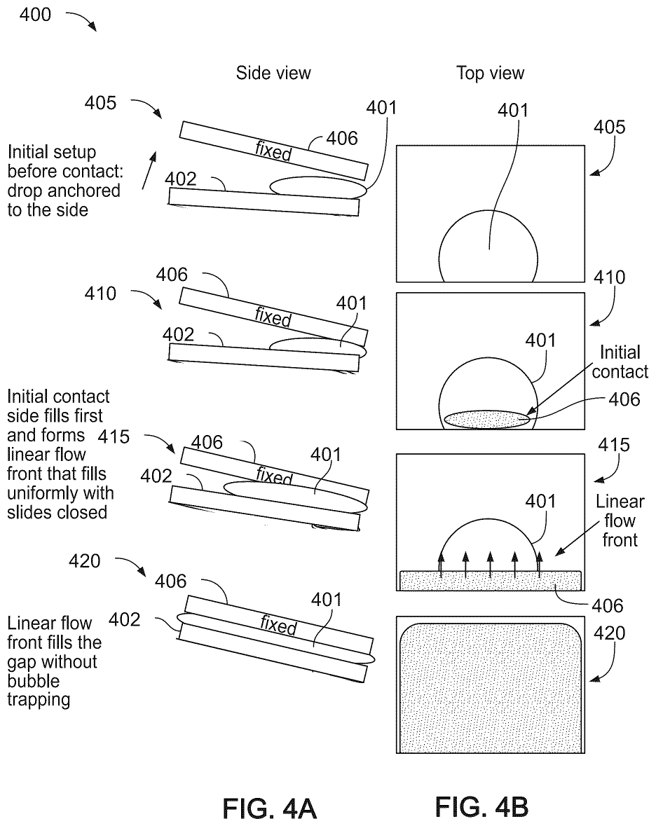

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or intermediate agents thereof (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as intermediate agents for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II) (d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663, which is herein incorporated by reference.

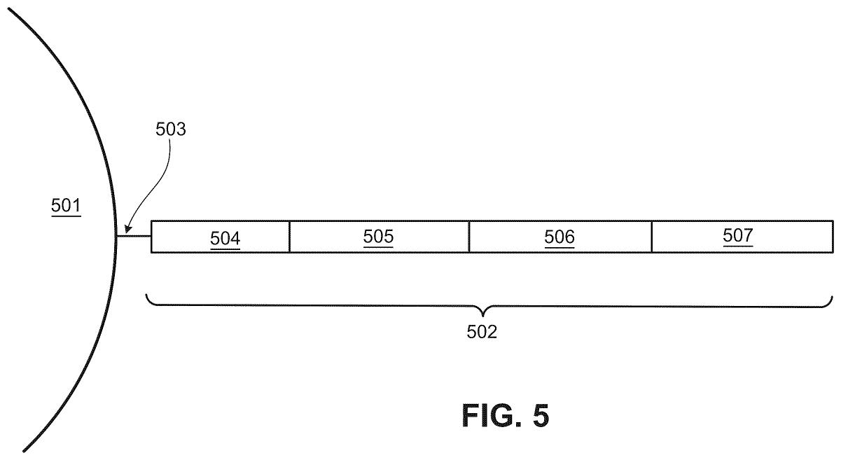

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

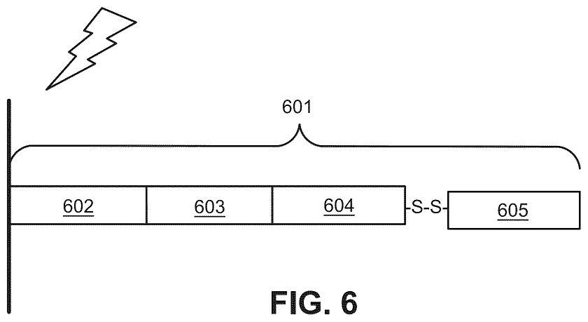

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

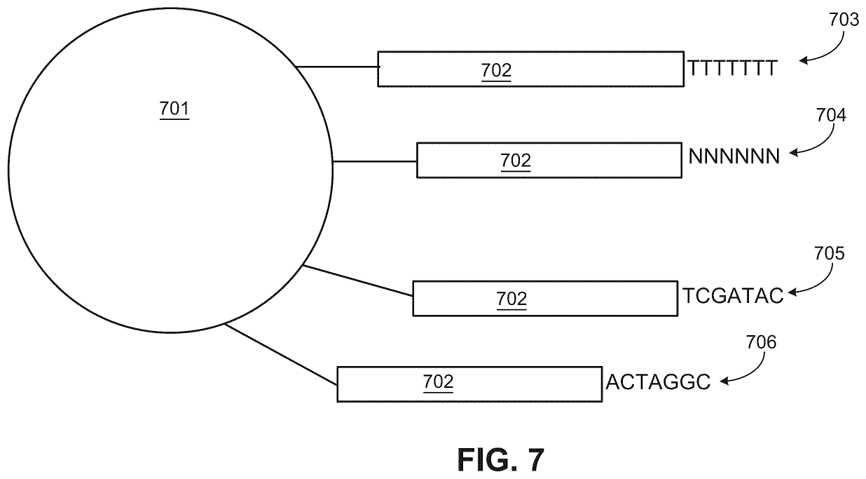

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

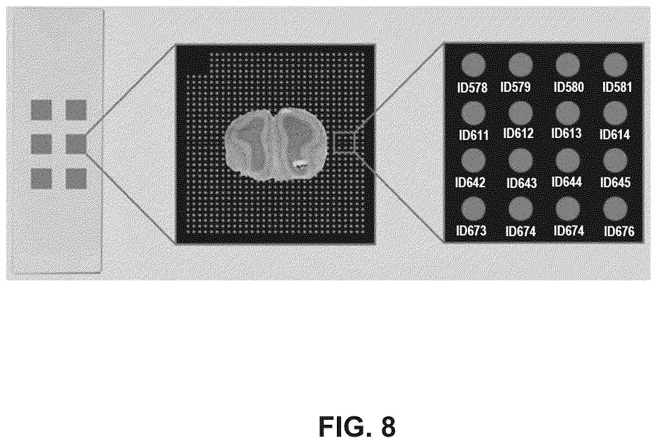

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No.

WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 August 21; 45(14): e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

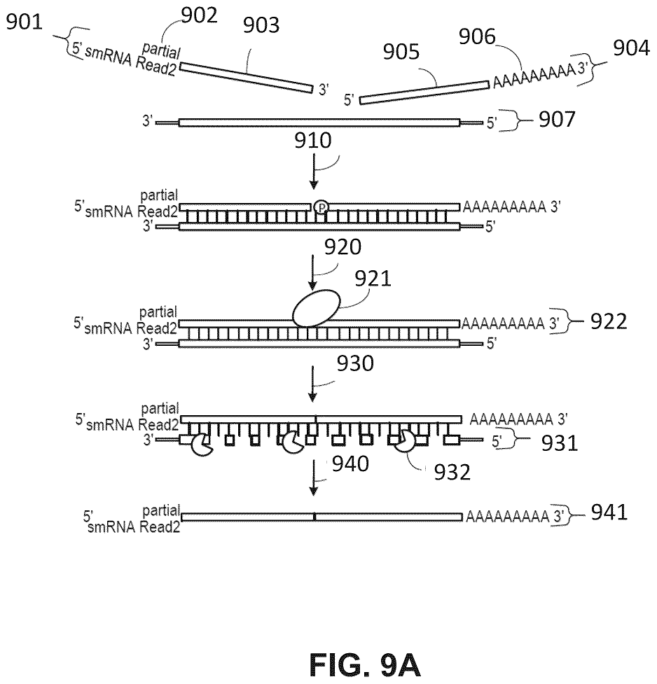

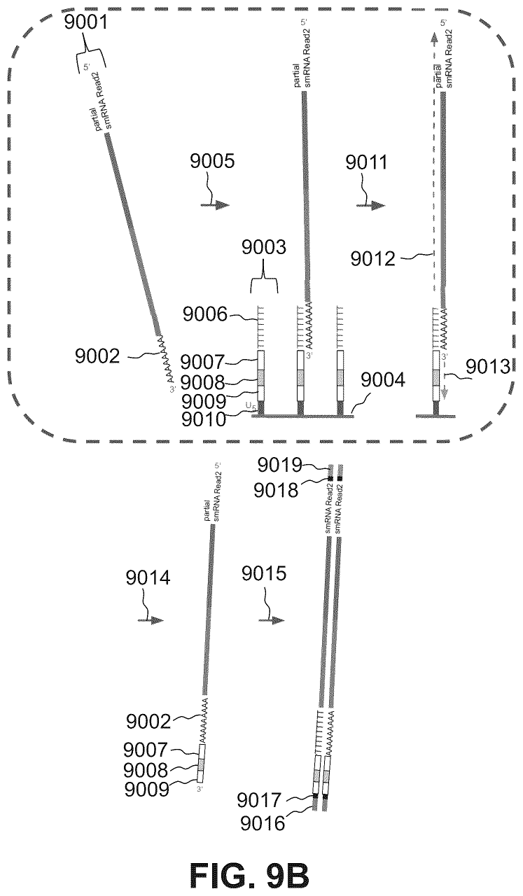

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

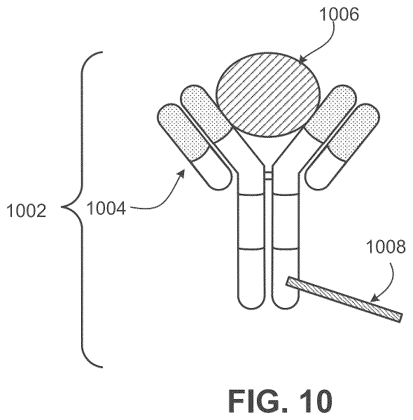

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

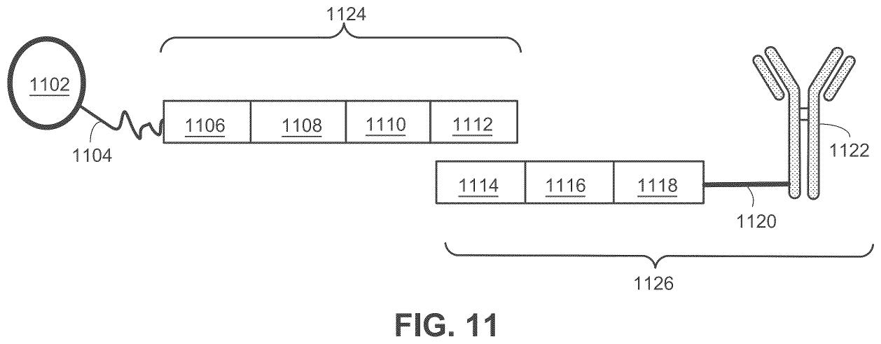

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Methods and Compositions for Analyte Localization and Sequencing

### 1. Overview of Methods for Analyte Localization and Sequencing

The present disclosure provides methods for determining the location of analytes or intermediate agents in biological samples. These methods utilize a sequencing array comprising multiple pores to facilitate the migration and sequencing of analytes, enabling high-resolution spatial and molecular analysis. The methods described herein can include any one of the biological samples described in part (A) of this disclosure.

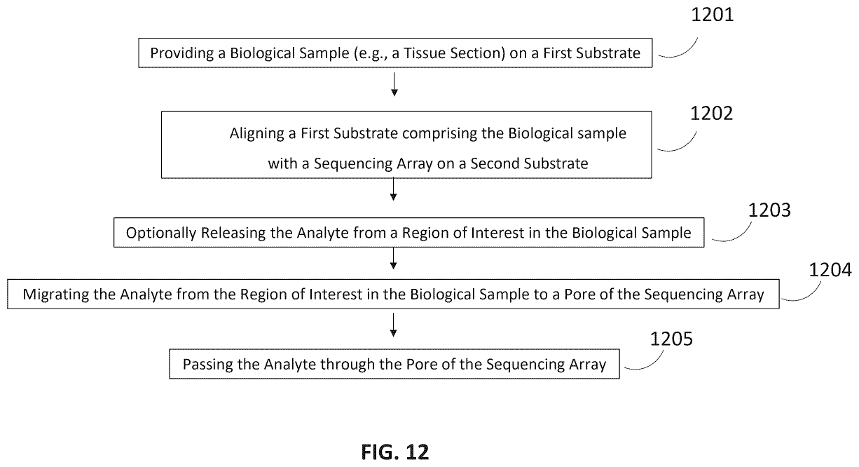

In some instances, referring to FIG. 12, the methods include determining a location of an analyte in a biological sample. In some instances, the methods include 1201 providing a biological sample (e.g., a tissue section) on a first substrate e.g., a slide. In some instances, the methods include 1202 aligning a first substrate comprising the biological sample with a sequencing array on a second substrate, wherein the sequencing array comprises a plurality of pores, such that at least a portion of the biological sample is aligned with at least a portion of the sequencing array on the second substrate. In some instances, the methods also include identifying a region of interest in the biological sample and/or 1203 optionally releasing the analyte from a region of interest in the biological sample. The methods can also include 1204 migrating the analyte from the biological sample to the sequencing array; and 1205 passing the analyte through a pore of the sequencing array, thereby sequencing the analyte, and using the sequence of the analyte and the location of the pore through which the analyte passed to determine the location of the analyte in the biological sample.

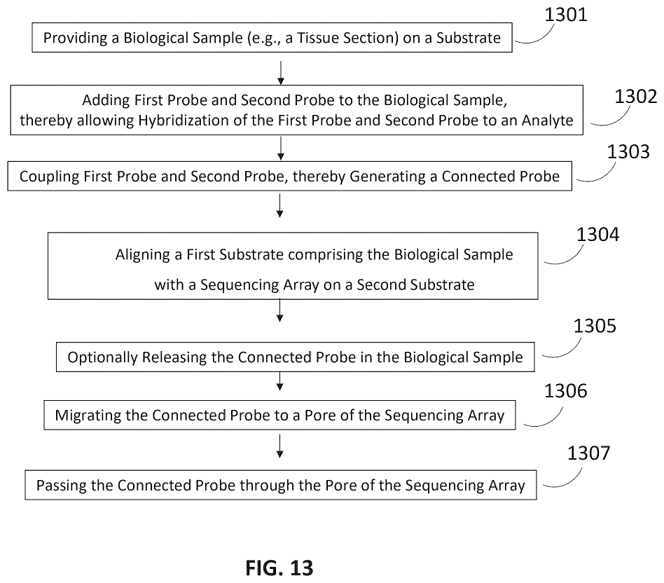

In some instances, the methods include determining a location of an intermediate agent (e.g., a connected probe or an oligonucleotide from an antibody-tagged oligonucleotide (e.g., an analyte capture agent as used herein)). Referring to FIG. 13, the methods include 1301 providing a biological sample (e.g., a tissue section) on a first substrate e.g., a slide; 1302 adding a first probe and a second probe to the biological sample, thereby allowing hybridization of the first probe and second probe to an analyte in the biological sample; 1303 coupling (e.g., ligating) the first probe and second probe, thereby generating a connected probe (e.g., a ligation product); 1304 aligning a first substrate comprising the biological sample with a sequencing array on a second substrate; 1305 optionally releasing the connected probe (e.g., ligation product) from the biological sample; 1306 migrating the connected probe (e.g., ligation product) to a pore of the sequencing array; and 1307 passing the connected probe (e.g., ligation product) through the pore of the sequencing array.

In some instances, either alone or in combination with nucleic acid analysis, spatial location of proteins/antigens can be determined. For example, when proteins alone are analyzed, steps 1302 and 1303 are omitted, and are replaced by steps of contacting analyte capture agents to the biological sample. This step is followed generally by steps 1304-1307. It is appreciated that the steps of protein and nucleic acid analysis can be performed at the same time on the same biological sample.

In some instances, the methods described herein include determining a location of an analyte or an intermediate agent in a biological sample. The methods can include (a) aligning a first substrate comprising the biological sample with a sequencing array on a second substrate, wherein the sequencing array comprises a plurality of pores, such that, in some embodiments, at least a portion of the biological sample is aligned with at least a portion of the sequencing array on the second substrate. After, the method includes (b) migrating the analyte or the intermediate agent from the biological sample to the sequencing array. Migrating can be active or passive. In some instances, migrating is performed using electrophoretic transfer of the analyte or intermediate agent from the first substrate to the second substrate. After migration (either active or passive), the methods include (c) passing the analyte or the intermediate agent through a pore of the sequencing array, thereby sequencing the analyte or the intermediate agent, and using the sequence of the analyte or the intermediate agent and the location of the pore through which the analyte or the intermediate agent passed to determine the location of the analyte or the intermediate agent in the biological sample.

In some instances, the methods include determining location of an analyte or an intermediate agent in a region of interest in a biological sample. In some instance, the methods include (a) aligning a first substrate comprising the biological sample with a sequencing array on a second substrate, wherein the sequencing array comprises a plurality of pores, such that at least a portion of the biological sample is aligned with at least a portion of the sequencing array on the second substrate; (b) releasing the analyte or the intermediate agent from the region of interest in the biological sample; (c) migrating the analyte or the intermediate agent from the region of interest in the biological sample to a pore of the sequencing array; and (d) passing the analyte or the intermediate agent through the pore of the sequencing array, thereby sequencing the analyte or the intermediate agent, and using the sequence of the analyte or the intermediate agent and the location of the region of interest in the biological sample to determine the location of the analyte or the intermediate agent in the region of interest in the biological sample.

In some instances, the methods include assaying an analyte in a region of interest in a biological sample. In some instance, the methods include (a) providing a sequencing array on a second substrate, wherein the sequencing array comprises a plurality of pores, aligning a first substrate comprising the biological sample with the sequencing array such that at least a portion of the biological sample is aligned with at least a portion of the sequencing array on the second substrate; (b) releasing the analyte, or an intermediate agent thereof, from the region of interest in the biological sample on the first substrate; (c) migrating the analyte or the intermediate agent thereof from the region of interest in the biological sample on the first substrate to the sequencing array on the second substrate; and (d) traversing the analyte or the intermediate agent thereof through a pore of the sequencing array to sequence the analyte or the intermediate agent thereof, and using the sequence of the analyte or the intermediate agent thereof and the location of the region of interest in the biological sample to determine the presence of the analyte in the region of interest in the biological sample.

In some embodiments, infrared light is used to release the analyte or intermediate agent from the region of interest in the biological sample. In some embodiments, the biological sample is exposed to or contacted with the infrared light. In some embodiments, exposure to infrared light locally increases the temperature of the biological sample in the region of interest, locally denatures polynucleotides (e.g., the analyte or intermediate agent thereof) present in the region of interest, and/or locally increases enzymatic activity in the region of interest.

Migrating in any one of the methods herein can be passive or active. During passive migration, an analyte or an intermediate agent thereof migrates directly from the biological sample to a proximal (e.g., closest) sequencing pore. When migration is passive, the location of the pore where the analyte or an intermediate agent thereof migrates can be used to determine the location of the analyte in the biological sample (e.g., the tissue section). In other methods, migration is active. Active migration can be performed by application of a stimulus such as using electrophoretic transfer. In this situation, any molecule released under the electric field can be transferred to the array.

Electrophoretic methods are widely used for the separation and transfer of nucleic acids, such as DNA and RNA, from one substrate to another (e.g., from the first substrate to the substrate having the sequencing array). These techniques utilize an electric field to drive the movement of charged molecules through media, allowing for the effective separation based on size and charge. Buffers that can be used in nucleic acid migration include Tris-acetate-EDTA (TAE) and Tris-borate-EDTA (TBE). In some instances, salts, such as sodium chloride (NaCl), are added to these buffers to maintain ionic strength thereby stabilizing the charge of nucleic acids and facilitating their migration through the media. In some instances, in the context of nucleic acid transfer from the first substrate to the sequencing array, conductive materials coated on both the first substrate and/or the second substrate can be used.

### 2. Substrates for the Biological Sample and for the Sequencing Array

The methods described herein can be performed using multiple substrates. Initially, a biological sample (e.g., a tissue section) is placed on a first substrate. In some instances, the first substrate is a glass substrate. In some instances, the first substrate is an electronically conductive substrate. In some instances, the electronically conductive substrate is a glass slide coated with Indium Tin Oxide (ITO), gold, carbon, silver, graphene, polyaniline, aluminum, or any suitable conductive polymer(s) or combination thereof.

The first substrate with a conductive layer is shown in FIG. 14. Referring to FIG. 14, a biological sample 1403 is placed on a conductive layer 1402 on a first substrate 1401. In some instances, the analyte or the intermediate agent is released. In some instances, the biological sample is removed from a hydrogel from which it had been embedded, and the analyte or the intermediate agent remains in the hydrogel 1403. Optionally, the biological sample can be removed from the first substrate, but the analyte or the intermediate agent (collectively 1404, shown after migration) remains in a hydrogel. In some instances, the second substrate 1405 comprises a conductive layer 1406. The conductive layer on the first and/or second substrate can be one or more of Indium Tin Oxide (ITO), gold, carbon, silver, graphene, polyaniline, aluminum, or any suitable conductive polymer(s).

The second substrate includes a series of pores that can be used to sequence an analyte or an intermediate agent thereof. In some instances, the second substrate includes at least 10 pores. In some instances, the second substrate includes at least 100 pores. In some instances, the second substrate includes at least 1,000 pores. In some instances, the second substrate includes at least 10,000 pores. At its core, pore sequencing technology utilizes a nanoscale pore through which nucleic acids pass. As the nucleic acid molecules traverse the pore, they induce changes in an electrical current that can be measured. These changes correspond to the specific nucleotides present in a nucleic acid strand, allowing for real-time sequencing of the nucleic acid molecule.

One of the key benefits of sequencing using pores on a substrate is its ability to produce long reads, often exceeding tens of thousands of base pairs. This is particularly advantageous for sequencing complex genomes, structural variants, and regions with repetitive sequences, which can be challenging for short-read technologies. Additionally, sequencing using pores on a substrate enables direct RNA sequencing, allowing researchers to analyze RNA modifications and splice variants without the need for reverse transcription to cDNA, preserving the original RNA structure and information.

In some instances, to translocate a nucleic acid through a pore (e.g., nanopore), voltage is applied to create an electric field. In some instances, as the nucleic acid strands pass through the nanopore, they disrupt the ionic current flowing through the pore. Each nucleotide causes a distinct change in the current, which is detected in real time. The changes in the electrical current are recorded and translated into nucleotide sequences. The duration and magnitude of the current change correspond to specific nucleotides (A, T, C, or G). This real-time data collection allows for continuous monitoring of the sequencing process. Then, in some instances, the recorded electrical signals are analyzed to determine the sequence of the nucleic acid analyte or its intermediate agent thereof. Sequencing using pores on a substrate have been described, for example, in Wang et al., Nature Biotechnology volume 39, pages1348-1365 (2021); Jain et al., Genome Biol. 2016 November 25; 17(1): 239, and at nanoporetech. com/documentation, each of which is incorporated by reference in its entirety.

In some instances, a flow cell is placed between the first substrate and the second substrate. In some instances, one or more adhesives connect the first substrate and the second substrate to the flow cell. Flow cells can include channels that permit reagents, solvents, features, and analytes (or their intermediate agents thereof) to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell.

In some instances, the methods disclosed include applying a mask to one of the substrates (i.e., the first substrate and/or the second substrate). In some embodiments, the mask is applied to a first substrate. In some instances, the mask is applied to the side of the substrate (e.g., the sequencing array substrate). As used here, in some embodiments, a mask is a composition that prevents the interaction between the nanopore sequencing array and the analytes or intermediate agents in the biological sample. In some embodiments, a mask is a composition that is placed on top of the biological sample.

A wide variety of different compositions can be used for the mask so long as it reduces or prevents the activation source (e.g., light) from activating (e.g., releasing) the analyte or an intermediate agent thereof from the biological sample. Exemplary materials for the mask include, but are not limited to, photoresists, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

The mask can prevent release of some of the analytes or their intermediate agents thereof from the biological sample. In doing so, the mask can take on any shape (regular, irregular, drawn, etc.). For instance, the mask can cover regions outside of a region of interest, thereby allowing light into a region of interest to promote release of analytes or the intermediate agents thereof.

In some instances, the first substrate or the second substrate can include a hydrogel. In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus. A hydrogel as described herein can include a cross-linked 3D network of hydrophilic polymer chains. A hydrogel subunit can be a hydrophilic monomer, a molecular precursor, or a polymer that can be polymerized (e.g., cross-linked) to form a three-dimensional (3D) hydrogel network. In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

In some instances, where the biological sample is a tissue section, the hydrogel can include a monomer solution and an ammonium persulfate (APS) initiator/tetramethylethylenediamine (TEMED) accelerator solution.

In some embodiments, a biological sample is embedded in a hydrogel to facilitate sample transfer to another location (e.g., to the sequencing array).

In some embodiments, the hydrogel is removed prior to migration of the analyte or an intermediate agent thereof. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample.

Biological samples embedded in hydrogels can be cleared using any suitable method. For example, electrophoretic tissue clearing methods can be used to remove biological macromolecules from the hydrogel-embedded sample. In some embodiments, a hydrogel-embedded sample is stored in a medium before or after clearing of hydrogel (e.g., a mounting medium, methylcellulose, or other semi-solid mediums). In some embodiments, the hydrogel chemistry can be tuned to specifically bind (e.g., retain) particular species of analytes (e.g., RNA, DNA, protein, etc.). In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE), Label-IT Amine and Label X (Chen et al., Nat. Methods 13: 679-684, (2016)). Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

Additional methods and aspects of hydrogel embedding of biological samples are described for example in Chen et al., Science 347(6221): 543-548, 2015, the entire contents of which are incorporated herein by reference.

### 3. Nanopore Sequencing

In some embodiments, sequencing an analyte or intermediate agent in accordance with the disclosed methods includes a pore-based sequencing approach. In a specific, embodiment sequencing an analyte or intermediate agent in accordance with the disclosed methods includes nanopore sequencing. Nanopore sequencing is one method of determining the sequence of polynucleotide molecules based on the property of physically sensing the individual nucleotides (or physical changes in the environment of the nucleotides (e.g., an electric current)) within an individual polynucleotide (e.g., DNA and RNA) as it traverses through a nanopore aperture. In principle, the sequence of a polynucleotide can be determined from a single molecule. However, in practice, typically a polynucleotide sequence be determined from a statistical average of data obtained from multiple passages of the same molecule or the passage of multiple molecules having the same polynucleotide sequence. The use of membrane channels to characterize polynucleotides as the molecules pass through the small ion channels has been studied by Kasianowicz et al. (Proc. Natl. Acad. Sci. USA. 93: 13770-13773, 1996, which is incorporated herein by reference) by using an electric field to force single stranded RNA and DNA molecules through a 1.5 nanometer diameter nanopore aperture (for example, an ion channel) in a lipid bilayer membrane. The diameter of the nanopore aperture permitted only a single strand of a polynucleotide to traverse the nanopore aperture at any given time. As the polynucleotide traversed the nanopore aperture, the polynucleotide partially blocked the nanopore aperture, resulting in a transient decrease of ionic current.

In some embodiments, the nanopore is embedded in a membrane, structure or an interface, which separates two media. As the polynucleotide (e.g., analyte or intermediate agent) passes through the nanopore, the polynucleotide alters an ionic current by blocking the nanopore. As the individual nucleotides pass through the nanopore, each base/nucleotide alters the ionic current in a manner that allows the identification of the nucleotide transiently blocking the nanopore, thereby allowing one to characterize the nucleotide composition of the polynucleotide and determine the nucleotide sequence of the polynucleotide. Individual nucleotides can be identified at the single molecule level from their current amplitude when they interact with the pore. The nucleotide is present in the pore (either individually or as part of a polynucleotide) if the current flows through the pore in a manner specific for the nucleotide (i.e. if a distinctive current associated with the nucleotide is detected flowing through the pore). Successive identification of the nucleotides in a target polynucleotide allows the sequence of the polynucleotide to be determined.

The sequencing methods may be carried out using any apparatus that is suitable for investigating a membrane/pore system in which a pore is inserted into a membrane. The method may be carried out using any apparatus that is suitable for transmembrane pore sensing. For example, the apparatus can include a chamber including a buffer (e.g., an aqueous solution) and a barrier that separates the chamber into two sections. The barrier has an aperture in which the membrane containing the pore is formed. In some embodiments, the sequencing is carried out using the apparatus described, e.g., in International Application No. PCT/GB08/000562. The methods can involve measuring the current passing through the pore during interaction with the nucleotide(s). Therefore, the systems or apparatus used for sequencing also can also include an electrical circuit capable of applying a potential and measuring an electrical signal across the membrane and/or pore. The methods may be carried out using a patch clamp or a voltage clamp.

In some embodiments, a chamber is formed between a first substrate (e.g., comprising a biological sample) and a second substrate comprising the pore array (sequencing array), and optionally one or more spacers. Media (e.g., containing permeabilization reagents, detergents, salts, ions, and/or buffers) can be disposed between the first and second substrate such that a potential difference could be applied between the first and second substrate.

The methods may be carried out using any suitable membrane (such as an amphiphilic layer or a lipid bilayer) system in which a pore is inserted into a membrane. The sequencing method can be carried out using (i) an artificial membrane (such as an amphiphilic layer or a lipid bilayer) comprising a pore, (ii) an isolated, naturally-occurring lipid bilayer comprising a pore, or (iii) a cell having a pore inserted therein.

In some embodiments, the nanopore of the sequencing array includes a protein nanopore, e.g., one formed by alpha-hemolysin (aHL) or engineered variants thereof in a planar lipid bilayer system. In some embodiments, the sequencing array is provided in a biochip formed by hydrogel-encapsulated lipid bilayer with a protein nanopore embedded therein or a micro-droplet bilayer system. Biochips and micro-droplet bilayer systems have been described (Shim and Gu; Stochastic Sensing on a Modular Chip Containing a Single-Ion Channel Anal. Chem. 2007, 79, 2207-2213; Bayley, H. et al. Droplet interface bilayers. Mol. Biosyst. 4, 1191-1208 (2008). In certain embodiments, the sequencing array includes a synthetic nanopore. Synthetic nanopores include, but are not limited to, nanopores comprising silicon nitride or graphene.

Suitable conditions for measuring ionic currents through transmembrane protein pores are known in the art. In some embodiments, pore-based sequencing is carried out with a voltage applied across the pore. The voltage used can be in the range of −400 mV to +400 mV. The voltage used can be in a range having a lower limit selected from −400 mV, −300 mV, −200 mV, 150 mV, −100 mV, −50 mV, −20 mV and 0 mV and an upper limit independently selected from +10 mV, +20 mV, +50 mV, +100 mV, +150 mV, +200 mV, +300 mV and +400 mV. It is possible to increase discrimination between different nucleotides by a pore by using an increased applied potential.

The sequencing portion of the disclosed methods can be carried out in the presence of any alkali metal chloride salt. In some embodiments, the salt is present in the solution in the chamber. Exemplary salts include Potassium chloride (KCl), sodium chloride (NaCl) or caesium chloride (CsCl). The salt concentration is typically from 0.1 to 2.5M, from 0.3 to 1.9M, from 0.5 to 1.8M, from 0.7 to 1.7M, from 0.9 to 1.6M or from 1M to 1.4M. The salt concentration is preferably from 150 mM to 1M. High salt concentrations provide a high signal to noise ratio and allow for currents indicative of the presence of a nucleotide to be identified against the background of normal current fluctuations.

The methods are typically carried out in the presence of a buffer. In the exemplary apparatus discussed above, the buffer is present in the aqueous solution in the chamber. Any buffer may be used in the method of the disclosure. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature.

The whole or only part of a polynucleotide may be sequenced as disclosed herein. The polynucleotide can be any length. In some embodiments, the disclosed methods allow sequencing polynucleotides with a size in the range of between 10 nucleotides to 50 thousand nucleotides. The number of nucleotides in the polyncuelotide can be 10, 15, 20, 25, 30, 35, 40, 45, 50, 75, 100, 150, 200, 250, 300, 350, 400, 450, 500, 750, 1,000, 1,500, 2,000, 2,500, 3,000, 3,500, 4,000, 4,500, 5,000, 10,000, 15,000, 20,000, 30,000, 40,000, 50,000 or any number therebetween. It may also be desirable to sequence polynucleotides in excess of 50,000 nucleotides.

In some embodiments, sequencing an analyte or intermediate agent includes (i) contacting analyte or intermediate agent with a pore within a sequencing pore array such that a nucleotide from the analyte or intermediate agent interacts with the pore, (ii) measuring the current passing through the pore during the interaction and thereby determining the identity of the nucleotide; and (iii) repeating steps (i) to (ii) to determine the sequence of the analyte or intermediate agent.

### 4. Release and Migration of an Analyte or its Intermediate Agent Thereof to the Sequencing Array

In some embodiments, an activation source can be applied to the biological sample in order to release the analytes or intermediate agents thereof from the biological sample (e.g., a tissue section). In some embodiments, after applying the mask to the distal side of the substrate containing the biological sample, an activation source can be applied to the biological sample in order to release the analytes or intermediate agents thereof from the biological sample (e.g., a tissue section). In some instances, the activation source is light. In some instances, the activation source is visible light. In some instances, the activation source is not visible light (e.g., infrared light or ultraviolet light). In some embodiments, a light source can release an analyte or an intermediate agent thereof with a photo-cleavable linker (e.g., a photo-sensitive chemical bond). Light sources can be targeted to a region of interest on the array (e.g., a digital micro-device). Thus, the disclosed methods include exposing the biological sample, or a region of interest therein, to ultraviolet light or infrared light.

In some embodiments, infrared light can serve as a heat source. In some embodiments, the biological sample is directly exposed or contacted with infrared light. In some embodiments, a reagent medium (applied to the biological sample) is exposed or contacted with infrared light. Exposure to infrared light may locally increase the temperature of the biological sample in the region of interest. This, in turn, can denature polynucleotide complexes (e.g., melt RNA/DNA; complexes of connected probes hybridized to target nucleic acid analytes). In some embodiments, exposure to infrared light may locally activate or increase enzymatic activity in the region of interest. This can be advantageous to release an intermediate agent locally from a region of interest, for example by activating or increasing activity of an RNase (e.g., RNase H) to release an intermediate agent, such as a connected probe or ligation product, from target RNA. In some embodiments, infrared light exposure can be used to both locally increase the temperature of the biological sample in the region of interest and locally activate or increase enzymatic activity in the region of interest.

In some embodiments, light-absorbing particles or molecules are included in the reagent media to release the analytes or intermediate agents in a region of interest from the biological sample. Such light-absorbing molecules include dyes and nanoparticles. Exemplary light-absorbing molecules include green light absorbing dyes that selectively absorb green light (around 500-570 nm) in the visible spectrum, such as, Bindschedler's Green, Malachite Green, and organic dyes such as BTD-DTP1-3. Exposure of the reagent media to the appropriate wavelength of light (e.g., green light) in a desired area can be used to selectively release an analyte or intermediate agent from a specific region of interest in the biological sample. In some embodiments, light-absorbing nanoparticles that absorb light of particular wavelengths are included in the reagent media. Examples of light-absorbing nanoparticles suitable for use in accordance with the disclosed methods include metal nanoparticles (e.g., Au, Ag, Cu, Zn, Al, Fe), and semiconductor nanoparticles (Si, cadmium sulfide, zinc oxide, CdS, CdSe, CdTe). Exposure of the reagent media having light-absorbing nanoparticles to the appropriate wavelength of light in a desired area can be used to selectively release an analyte or intermediate agent from a specific region of interest in the biological sample.

In some embodiments, a heat source can release an analyte or an intermediate agent thereof with a heat-cleavable linker. In some embodiments, an analyte or an intermediate agent thereof is released by applying heated reagent medium of at least 50° C., e.g., at least about 50° C., 60° C., 70° C., 80° C., or 90° C. In some instances, the activation source is heat, and the mask prevents heat from permeating through it to interact with the analyte or an intermediate agent thereof. In some embodiments, a cleavage domain in the analyte or intermediate agent thereof can be an ultrasonic cleavage domain. For example, ultrasonic cleavage can depend on nucleotide sequence, length, pH, ionic strength, temperature, and the ultrasonic frequency.

In some instances, light is used to release the analyte or the intermediate agent from a region of interest in the biological sample. In some instances, the light is ultraviolet light. Ultraviolet (UV) light is categorized into several wavelength ranges, each with distinct characteristics and applications. The UV spectrum is generally divided into three main regions: UVA, UVB, and UVC. UVA (315-400 nm) has the longest wavelengths; UVB (280-315 nm) has medium wavelengths; UVC (100-280 nm) possesses the shortest wavelengths. In some instances, the UV light is UVA. In some instances, the UV light is UVB. In some instances, the UV light is UVC light. It is also appreciated that the UV light can range among UVA, UVB, and UVC light.

The releasing step can include exposing multiple regions of interest in the biological sample to light. The methods described herein allow for analysis of multiple regions of interest within the biological sample. For example, a mask can be added to the first substrate. After, the sample on the first substrate is treated with UV light. After UV treatment (or any stimuli described herein), the analyte or an intermediate agent thereof migrates to the second substrate that comprises the sequencing array. After sequencing the analyte(s) in the area not masked, the mask can be moved and the process can be repeated in a second region of interest. It is appreciated that this process can be repeated multiple (e.g., at least 2, 3, 4, 5, 6, 7, 8, 9, 10 or more times on a single biological sample. This multiplexing capability enhances throughput, enabling researchers to gather extensive data on various analytes within a single experiment.

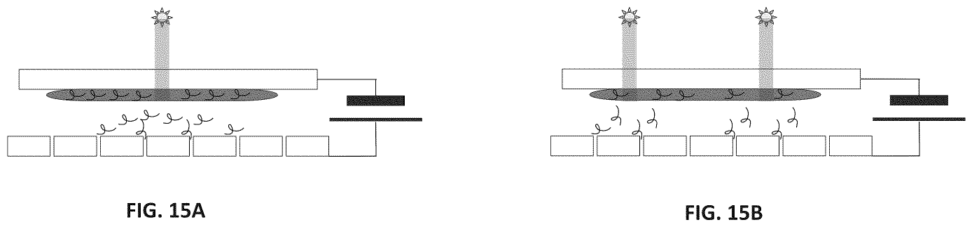

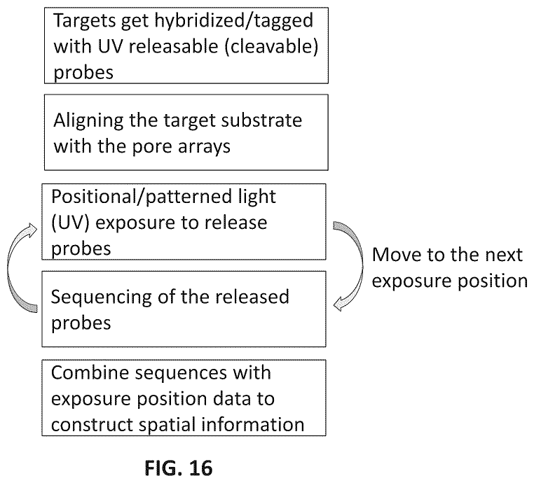

Once the analytes or intermediate agents are released (e.g., from the biological sample or hydrogel), they migrate to the second substrate. In some instances, analytes or intermediate agents from each region of interest in multiple regions of interest do not laterally diffuse to other regions of interest. Multiple regions of interest in the biological sample can include at least 2, 3, 4, 5, 6, 7, 8, 9, 10, or more regions. It is appreciated that the methods described herein can be sequentially performed in multiple regions of interest (e.g., one at a time). Then, the steps are repeated at a second region. As shown in FIG. 15A, one region of interest can be identified. Then, that region of interest can be exposed to a stimulus that releases the analytes in that region of interest. In some instances, the analytes then migrate to the second substrate, where they pass through any one of the nanopores on the second substrate (e.g., providing the sequence of the analyte or intermediate agent). In some instances, as shown in FIG. 15B, multiple regions of interest can be examined at the same time. Alternatively, sequential analysis can be performed. Referring to FIG. 16, targets get hybridized/tagged with UV releasable (cleavable) probes followed by aligning the substrate containing the biological sample with the substrate comprising the array having a plurality of pores; positional/patterned light (UV) exposure releases the probes; followed by sequencing of the released probes; and then determined sequences are combined with exposure position data to construct spatial information.

The methods described herein include aligning the two substrates. In some instances, a first substrate containing a biological sample is aligned with a second substrate featuring a sequencing array that includes a plurality of pores. Alignment can include bringing the two substrates into contact or proximity such that a first side of the first substrate containing the biological sample is positioned opposite a first side of the second substrate containing the array comprising a plurality of pores. This alignment ensures that at least a portion of the biological sample directly overlaps with the sequencing array, facilitating efficient analyte or intermediate agent transfer and retention of the spatial information of the overlapped portion.

After aligning, analytes or intermediate agents migrate from the first substrate to the second substrate. The method involves migrating the analyte or intermediate agent from the biological sample to the sequencing array. Migration can be driven by various mechanisms. In some instances, the mechanism is via application of an electric field. In these instances, an electric field is applied across the first and second substrates, promoting the movement of charged analytes toward the sequencing array. In some instances, the mechanism utilizes chemical agents. For instance, the methods use releasing enzymes (e.g., RNases, DNases) or other chemical mediators can enhance analyte release and migration. In another example, the methods use light, (e.g., ultraviolet light) to induce the release of analytes from specific regions of interest within the biological sample. In some embodiments, the biological sample is heated to release the analytes or intermediate agents thereof to allow migration to occur. For example, the biological sample can be heated above a melting temperature (Tm) or annealing temperature of an intermediate agent (e.g., a connected probe, cDNA) to free (e.g., separate) it from the analyte so it can migrate to the sequencing array to be analyzed. A melting temperature can range from about 45° C. to about 70° C. (e.g., about 45° C., about 50° C., about 55° C., about 60° C., about 65° C., or about 70° C.). These temperatures can vary depending on the length and content of the analyte.

In some instances, analyte or intermediate agent release (and thus migration) is controlled by varying different parameters. For example, in some instances, enzyme concentration can vary by adjusting the concentration of releasing enzymes to modulate the rate of analyte or intermediate agent release. In some instances, analyte or intermediate agent release is altered by changing the temperature of the first substrate to influence release (and/or migration) dynamics. In some instances, modifying the intensity and wavelength of light is used for analyte release.

After migration, the analytes or intermediate agents are sequenced by passing them through the pores on the second substrate. For example, following migration, the analyte or intermediate agent is passed through a pore of the sequencing array. As the analyte traverses the pore, it generates a unique electronic signature that is captured for sequencing. This step is crucial for determining both the identity of the analyte and its precise location based on the corresponding pore. The sequencing data obtained is utilized to determine the specific location of the analyte within the biological sample. The sequence of the analyte, coupled with the known location of the pore through which it passed, provides information about the spatial distribution of the analyte. Additionally, the method can assess the expression levels of analytes in defined regions of interest.

The methods can be further enhanced by hybridizing specific probes to the analytes prior to sequencing. The probes, which are complementary to target nucleic acid analytes, facilitate accurate detection and quantification. Subsequent ligation of probes enhances specificity and allows for the identification of specific mutations or variants in the analytes.

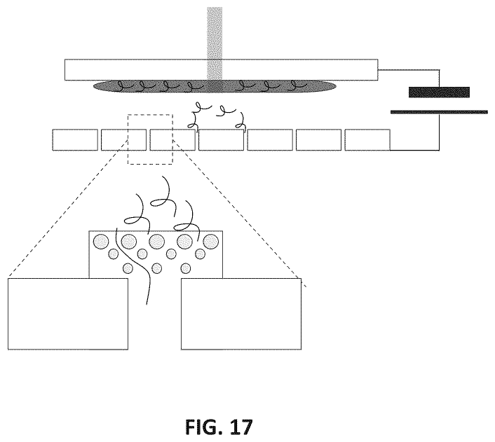

In some instances, as shown in FIG. 17, the second substrate (e.g., having the sequencing array) includes measures to mitigate or suppress diffusion of an analyte or an intermediate agent thereof through a pore of a sequence array. For example, in some embodiments, a physical structure can be added to the sequencing array to mitigate or suppress diffusion. In some embodiments, the physical structure can include additional channels, wells, projections, ridges, or divots that are placed on the second substrate (e.g., the substrate comprising the sequencing array) that prevents or limits diffusion of an analyte or intermediate agent. In another example, each pore on the sequencing array can be treated at its pillar region with one or more of these physical structures. In nanopore sequencing arrays, the pillar region refers to the structural component that enhances the functionality of the sequencing array device. Typically, the sequencing arrays include multiple pores embedded in a substrate, which is often designed with “pillars” or posts that support the nanopore structure. A pore having structures that make up the pillar region above each pore is shown in FIG. 17, where each circle represents structures that surround the pore. When a pillar region is used, the pillar region reduces crowding of analytes or intermediate agents inside of the pores compared to a sequencing array without the pillar region.

The methods may include steps for staining the biological sample (e.g., with immunofluorescent or histological dyes), followed by imaging to provide additional context for the spatial distribution of analytes. In some instances, the methods include staining the biological sample using immunofluorescence, immunohistochemistry, hematoxylin, and/or eosin staining. In some instances, the methods include imaging the biological sample.

### 5. Nucleic Acids and Proteins (or Their Intermediate Agents Thereof)

In some instances, the methods include direct spatial analysis of nucleic acid analytes. In some instances, the analyte is RNA. In some instances, the analyte is mRNA. In some instances, the analyte is DNA. In some instances, the analyte is genomic DNA or cDNA. When the analyte is directly analyzed, the analyte itself migrates from the first substrate to the second substrate as described in this application.

In some instances, RNA is converted to cDNA in the biological sample (e.g., in situ). To convert RNA to cDNA, a buffer mixture comprising a reverse transcriptase enzyme, deoxynucleotide triphosphates (dNTPs), primers (e.g., an oligo(dT) primer (for mRNA), a random hexamer, or a gene-specific primer) and, optionally, one or more salts and stabilizing agents, is added to the biological sample. The biological sample is heated to a suitable temperature (e.g., around 37-50° C.) to promote the reverse transcription reaction. Afterwards, in some instances, RNAse can be added to digest any remaining RNA. After generation of cDNA molecules, these cDNA molecules can migrate to the second substrate to the sequencing pores as described herein.

In some instances, the methods provided herein analyze nucleic acids or proteins using an intermediate agent thereof (also called a proxy or a proxy molecule). In some instances, the intermediate agent can include amplified DNA and/or cDNA, as described above. In instances where nucleic acid analytes are analyzed using an intermediate agent thereof, the methods can utilize probe pairs (or probe sets; the terms are interchangeable). In some embodiments, the probes included in the probe sets are nucleic acid probes. In some instances, the probe pairs are designed so that each probe in a probe pair hybridizes to a sequence in a nucleic acid analyte that is specific to the nucleic acid analyte (e.g., compared to the entire genome). That is, in some instances, a single probe pair can be specific to a single nucleic acid analyte.

In other embodiments, probes can be designed so that one of the probes of a pair is a probe that hybridizes to a specific sequence. Then, the other probe can be designed to detect a mutation of interest. Accordingly, in some instances, multiple second probes can be designed and can vary so that each hybridizes to a specific sequence. For example, one second probe can be designed to hybridize to a wild-type sequence, and another second probe can be designed to detect a mutated sequence. In some instances, the mutation includes an insertion, a deletion, a substitution, or a splicing error. Thus, in some instances, a probe set can include one first probe and two second probes (or vice versa).

In some embodiments, probe sets target all or nearly all of a genome (e.g., human genome). In instances where probe sets are designed to target an entire genome (e.g., the human genome), the methods disclosed herein can detect analytes in an unbiased manner. In some instances, one probe pair is designed to target one analyte (e.g., transcript). In some instances, more than one probe pair (e.g., a probe pair comprising a first probe and a second probe) is designed to target one analyte (e.g., transcript). For example, two, three, four, five, six, seven, eight, nine, ten, or more probe sets can be used to hybridize to a single analyte. Factors to consider when designing probes include presence of variants (e.g., SNPs, mutations) or multiple isoforms expressed by a single gene. In some instances, the probe pair does not hybridize to the entire analyte (e.g., a transcript), but instead the probe pair hybridizes to a portion of the entire analyte (e.g., transcript).

In some instances, about 5000, 10,000, 15,000, 20,000, or more probes pair (e.g., a probe pair comprising a first probe and a second probe) are used in the methods described herein. In some instances, about 20,000 probes pair are used in the methods described herein. In some embodiments, analyte analysis using the methods disclosed herein allows for examination of a subset of RNA analytes from the entire transcriptome. In some embodiments, the subset of analytes includes an individual target RNA. In some embodiments, the subset of analytes includes two or more targeted RNAs. In some embodiments, the subset of analytes includes one or more mRNAs transcribed by one or more targeted genes. In some embodiments, the subset of analytes includes one or more mRNA splice variants of one or more targeted genes. In some embodiments, the subset of analytes includes non-polyadenylated RNAs in a biological sample. In some embodiments, the subset of analytes includes detection of mRNAs having one or more single nucleotide polymorphisms (SNPs) in a biological sample.

In some embodiments, the subset of analytes includes mRNAs that mediate expression of a set of genes of interest. In some embodiments, the subset of analytes includes mRNAs that share identical or substantially similar sequences, which mRNAs are translated into polypeptides having similar functional groups or protein domains. In some embodiments, the subset of analytes includes mRNAs that do not share identical or substantially similar sequences, which mRNAs are translated into proteins that do not share similar functional groups or protein domains. In some embodiments, the subset of analytes includes mRNAs that are translated into proteins that function in the same or similar biological pathways. In some embodiments, the biological pathways are associated with a pathologic disease. For example, targeted RNA capture can detect genes that are overexpressed or underexpressed in cancer.

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 analytes. In some instances, the methods disclosed herein can detect the location of at least 5,000, 10,000, 15,000, 20,000, or more different analytes.

In some instances, the probes are DNA probes. In some instances, the probes are diribonucleotide-containing probes. In some embodiments, the first probe and/or the second includes ribonucleotides, deoxyribonucleotides, and/or synthetic nucleotides that are capable of participating in Watson-Crick type or analogous base pair interactions. In some embodiments, the first probe and/or the second probe includes deoxyribonucleotides. In some embodiments, the first probe and/or the second probe includes deoxyribonucleotides and ribonucleotides. In some embodiments, the first probe and/or the second probe includes a deoxyribonucleic acid that hybridizes to an analyte, and includes a portion of the oligonucleotide that is not a deoxyribonucleic acid. For example, in some embodiments, the portion of the first oligonucleotide that is not a deoxyribonucleic acid is a ribonucleic acid or any other non-deoxyribonucleic acid nucleic acid as described herein. In some embodiments where the first probe and/or the second probe includes deoxyribonucleotides, hybridization of the first probe and/or the second probe to the analyte (e.g., an mRNA molecule) results in a DNA: RNA hybrid. In some embodiments, the first probe and/or the second probe includes only deoxyribonucleotides and hybridization of the first probe and/or the second probe to the mRNA molecule results in a DNA: RNA hybrid.

In some instances, the first probe and the second probe hybridize to a nucleic acid analyte of at least about 25 to 100 nucleotides in length. In some instances, the first probe and/or the second probe is a DNA probe. In some embodiments, the method includes a first probe and/or the second probe that includes one or more sequences that are substantially complementary to one or more sequences of an analyte. In some embodiments, a first probe and/or the second probe includes a sequence that is about 10 nucleotides to about 100 nucleotides. In some embodiments, a sequence of the first probe and/or the second probe that is substantially complementary to a sequence in the analyte includes a sequence that is about 5 nucleotides to about 50 nucleotides. In some embodiments, a first probe and/or the second probe includes at least two ribonucleic acid bases (ribonucleotides) at the 3′ end. In such cases, a second probe comprises a phosphorylated nucleotide at the 5′ end. In some embodiments, a first probe and/or the second probe includes at least three, at least four, at least five, at least six, at least seven, at least eight, at least nine, or at least ten ribonucleic acid bases at the 3′ end.

In some embodiments, the methods include 2, 3, 4, or more probes. In some embodiments, each of the probes includes ribonucleotides, deoxyribonucleotides, and/or synthetic nucleotides that are capable of participating in Watson-Crick type or analogous base pair interactions. In some embodiments, each of the probes includes deoxyribonucleotides. In some embodiments, each of the probes includes deoxyribonucleotides and ribonucleotides. In some instances, the multiple probes span different target sequences, and multiple, serial ligation steps are carried out to determine the location and/or abundance of an analyte.

In some embodiments, the methods provided herein include hybridizing a first probe and a second probe (e.g., a probe pair). In some instances, the first and second probes each include sequences that are substantially complementary to one or more sequences (e.g., one or more target sequences) of an analyte of interest. In some embodiments, the first probe and the second probe bind to complementary sequences that are completely adjacent (i.e., no gap of nucleotides) to one another or are on the same transcript. In some instance, the first probe and the second probe hybridize to sequences that are at least 1, 2, 3, 4, 5, 6, 7, 8, 9, 10, or more nucleotides away from one another. In some instance, the methods include generating an extended first probe, wherein the extended first probe comprises a sequence complementary to a sequence between the sequence hybridized to the first probe and the sequence hybridized to the second probe. In some instance, the methods include coupling (e.g., ligating) the extended first probe and the second probe, for example, using a ligase selected from a PBCV-1 DNA ligase, a Chlorella virus DNA ligase, a single stranded DNA ligase, or a T4 DNA ligase. In some instances, the methods include generating an extended second probe, wherein the extended second probe comprises a sequence complementary to a sequence between the sequence hybridized to the first probe and the sequence hybridized to the second probe.

In some instance, the methods include coupling (e.g., ligating) the first probe and the extended second probe, for example, using a ligase selected from a PBCV-1 DNA ligase, a Chlorella virus DNA ligase, a single stranded DNA ligase, or a T4 DNA ligase. In some instances, ligation can be performed enzymatically or chemically, as described herein. Ligation can be performed using any of the methods described herein. In some embodiments, the step includes ligation of the first probe and the second probe, forming a ligation product. In some embodiments, a third oligonucleotide serves as an oligonucleotide splint to facilitate ligation of the first oligonucleotide (e.g., the first probe) and the second oligonucleotide (e.g., the second probe). In some embodiments, ligation is chemical ligation. In some embodiments, ligation is enzymatic ligation. In some embodiments, the ligase used for enzymatic ligation is a T4 RNA ligase (Rnl2), a splintR ligase, a single stranded DNA ligase, or a T4 DNA ligase. In some instances, the ligation is an enzymatic ligation reaction, using a ligase (e.g., T4 RNA ligase (Rnl2), a Chlorella virus DNA ligase, a single stranded DNA ligase, or a T4 DNA ligase). See, e.g., Zhang et al.; RNA Biol. 2017; 14(1): 36-44, which is incorporated by reference in its entirety, for a description of KOD ligase. Following the enzymatic ligation reaction, the probes (e.g., a first probe, a second probe) may be considered ligated.

In some embodiments, after generating a connected probe (e.g., ligation product), the connected probe (e.g., ligation product) is released from the analyte. In some embodiments, a connected probe (e.g., ligation product) is released from the analyte using an endoribonuclease. In some embodiments, the endoribonuclease is RNase H, RNase A, RNase C, or RNase I. In some embodiments, the endoribonuclease is RNase H. RNase H is an endoribonuclease that specifically hydrolyzes the phosphodiester bonds of RNA, when hybridized to DNA. In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNAse HI from E. coli, or RNase HII from E. coli. In some instances, the connected probe (e.g., ligation product) is released from the analyte using heat. In some instances, the temperature of the substrate containing the biological sample is heated to at least 50° C., at least 55° C., at least 60° C., at least 65° C., at least 70° C., at least 75° C., at least 80° C., or higher. In some instances, the biological sample is heated at one or more of these temperatures for at least 10 minutes, at least 30 minutes, at least an hour, or longer.

In some instances, the releasing step occurs before the permeabilization step. In some instances, the releasing step occurs after the permeabilization step. In some instances, the releasing step occurs at the same time as the permeabilization step (e.g., in the same buffer).

Methods of templated ligation are disclosed in PCT Publ. No. WO 2021/133849 and U.S. Pat. No. 11,332,790, each of which are incorporated by reference.

In some embodiments, the methods provided herein utilize analyte capture agents for spatial detection. An analyte capture agent refers to a molecule that interacts with a target analyte (e.g., a protein). Such analyte capture agents can be used to identify the analyte. In some embodiments, the analyte capture agent can include an analyte binding moiety and a capture agent barcode domain. In some embodiments, the analyte capture agent includes a linker. The linker can couple or connect the analyte binding moiety to the capture agent barcode domain. In some embodiments, the linker is a cleavable linker. In some embodiments, the cleavable linker is a photo-cleavable linker, a UV-cleavable linker, or an enzyme cleavable linker.

An analyte binding moiety is a molecule capable of binding to a specific analyte. In some embodiments, the analyte binding moiety comprises an antibody or antibody fragment. The antibody can include, without limitation, a monoclonal antibody, recombinant antibody, synthetic antibody, a single domain antibody, a single-chain variable fragment (scFv), and or an antigen-binding fragment (Fab). In some embodiments, the analyte binding moiety comprises a polypeptide and/or an aptamer. In some embodiments, the analyte is a protein (e.g., a protein on a surface of a cell or an intracellular protein) or other antigen.

In some embodiments, the capture agent barcode domain comprises an analyte binding moiety barcode. In some embodiments, the capture agent barcode domain comprises an analyte binding moiety barcode and does not comprise a capture handle sequence. In some embodiments, the capture agent barcode domain comprises an analyte binding moiety barcode and a capture handle sequence. In some embodiments, the capture handle sequence includes a poly (A) tail. The analyte binding moiety barcode refers to a barcode that is associated with or otherwise identifies the analyte binding moiety. Other embodiments of an analyte capture agent useful in spatial analyte detection are described herein.

In some embodiments, the capture agent barcode domain is released from the analyte binding moiety by using a different stimulus that can include, but is not limited to, a proteinase (e.g., Proteinase K), an RNase, and UV light.

In some instances, two analytes (e.g., two different antigens or proteins) in a biological sample are detected by a first analyte-binding moiety and a second analyte-binding moiety, respectively. In some embodiments, a first analyte-binding moiety and/or the second analyte-binding moiety is comprised in an analyte capture agent (e.g., any of the exemplary analyte capture agents described herein). In some embodiments, the first analyte-binding moiety and/or the second analyte-binding moiety is a first protein. In some embodiments, the first analyte-binding moiety and/or the second analyte-binding moiety is an antibody. For example, the antibody can include, without limitation, a monoclonal antibody, recombinant antibody, synthetic antibody, a single domain antibody, a single-chain variable fragment (scFv), and or an antigen-binding fragment (Fab). In some embodiments, the first analyte-binding moiety binds to a cell surface analyte (e.g., any of the exemplary cell surface analytes described herein).

In some embodiments, the first analyte-binding moiety and the second analyte-binding moiety each bind to the same analyte. In some embodiments, the first analyte-binding moiety and/or second analyte-binding moiety each bind to a different analyte. For example, in some embodiments, the first analyte-binding moiety binds to a first polypeptide and the second analyte-binding moiety binds to a second polypeptide.

In some embodiments of any of the methods of determining a location of at least one analyte in a biological sample, a first and/or a second oligonucleotide are bound (e.g., conjugated or otherwise attached using any of the methods described herein) to a first analyte-binding moiety and/or a second analyte-binding moiety, respectively.

### 6. Kits and Compositions

The present disclosure encompasses kits and compositions designed for implementing the methods of locating and sequencing analytes within biological samples as described in the claims. These kits and compositions facilitate efficient and effective analysis of biological samples, ensuring that users can easily conduct experiments involving analyte release, migration, and sequencing. In some instances, the kits and compositions include a first substrate.

In some instances, the kits and compositions include a first substrate designed to hold a biological sample. This substrate can be made from materials compatible with various biological assays, such as glass or polymer substrates, which may include a conductive layer to enhance signal detection.

In some instances, the kits and compositions include a second substrate with sequencing array. In some instances, the second substrate includes a sequencing array comprising a plurality of pores is provided. This array is engineered to facilitate the passage of analytes for sequencing, allowing for high-throughput analysis. Each pore is designed to optimize the electronic signature detection of the analytes as they pass through. In some instances, the kits and compositions can include a flow cell that can be placed between the first and second substrates to enhance the migration of analytes and control the fluid dynamics during the assay process.

In some instances, the kits and compositions include a light source, such as a UV lamp, may be included to assist in the controlled release of analytes from the biological sample, enabling precise targeting of regions of interest.

In some instances, the kits and compositions include a masking agent. In some instances, the masking agent can be provided to inhibit the release of analytes from non-target regions of the biological sample, ensuring that only the desired areas are analyzed.

The kits and compositions can contain a variety of reagents necessary for the release and sequencing of analytes, including: enzymes: Such as RNases (e.g., RNase A, RNase H) and DNases to facilitate the release and degradation of nucleic acids; polymerases: Including DNA and RNA polymerases to assist in the amplification of target sequences; ligation enzymes as described herein; templated ligation probes; analyte capture agents; permeabilization reagents such as proteases (e.g., proteinase K, trypsin) or surfactants, to enhance the permeability of the biological sample and improve analyte release.

The kits and compositions can also include compositions tailored for specific applications, such as hybridization buffers, ligation buffers, and solutions for analyte detection (e.g., for immunofluorescent or histological staining).

In some instances, the kits include detailed instructions for conducting the methods outlined in the claims, guiding users through the process of preparing the biological sample, aligning substrates, releasing analytes, and performing sequencing.

### EXAMPLES

### Example 1. Methods for Sequencing an Analyte Using Multiple Substrates

A tissue section is placed on an electronically conductive substrate, and the tissue section is prepared for spatial analysis. In brief, the tissue section is deparaffinized, H&E stained, and imaged. Next, the tissue section is hematoxylin-destained with a series of HCl solution washes. The section is then decrosslinked and incubated in PBS-Tween. In some embodiments, the electronically conductive substrate is a glass slide coated with Indium Tin Oxide (ITO).

The substrate having the biological sample is then aligned with a sequencing array on a second substrate so that some or all of the tissue sample aligns with at least some or all of the sequencing array. The sequencing array on the second substrate can have hundreds or thousands (e.g., 100; 1,000; 5,000; 10,000; 15,000) of pores. After aligning the glass slide with the second substrate, nucleic acid analytes (e.g., mRNA molecules) migrate from the tissue section to the sequencing array, where each analyte passes through a pore of the sequencing array, in order to sequence the analyte. Based on the location of the pore through which the analyte passes, the analyte's location in the tissue section can be determined.

### Example 2. Methods for Sequencing a Connected Probe (e.g., a Ligation Product) Using Multiple Substrates

A tissue section is placed on an electronically conductive substrate, and the section is prepared for spatial analysis. In some embodiments, the tissue section is deparaffinized, H&E stained, and imaged. Next, the tissue section is hematoxylin-destained with a series of HCl solution washes. The section is then decrosslinked and incubated in PBS-Tween. In some embodiments, the electronically conductive substrate is a glass slide coated with Indium Tin Oxide (ITO).

Individual probes (e.g., a first probe, a second probe) of probe pairs are hybridized to sequences of an analyte (e.g., an RNA molecule) in the tissue section. The probes then are ligated together, thereby creating a connected probe (e.g., a ligation product). The tissue section is treated with RNAse H to digest the RNA molecule, leaving a single stranded connected probe at the location of the analyte.

The substrate having the biological sample (e.g., glass slide) is then aligned with a sequencing array on a second substrate so that some or all of the tissue sample aligns with at least some or all of the sequencing array. The sequencing array on the second substrate can have hundreds or thousands (e.g., 100; 1,000; 5,000; 10,000; 15,000) of pores. After sandwiching the glass slide with the second substrate, the connected probes migrate from the tissue section to the sequencing array, where each connected probe passes through a pore of the sequencing array. Based on the location of the pore through which the connected probe passes, the analyte's location in the tissue section can be determined.

### Example 3. Methods for Sequencing a Proxy of a Protein Using Multiple Substrates

A tissue section is placed on a glass slide, and the section is prepared for spatial analysis, for example as described in Examples 1 and 2.

The tissue section is incubated with oligonucleotide-tagged antibodies targeting proteins of interest, followed by washing. The antibodies are tagged with oligonucleotides that have a barcode sequence that uniquely identifies the antibody and/or the target protein.

The glass slide is then aligned with a sequencing array on a second substrate so that some or all of the tissue section aligns with at least some or all of the sequencing array. The sequencing array on the second substrate can have hundreds or thousands (e.g., 100; 1,000; 5,000; 10,000; 15,000) of pores. After aligning the glass slide with the second substrate, the antibody associated oligonucleotides are released (e.g., using a reagent medium that includes RNase and Proteinase K) and migrate from the tissue section to the sequencing array, where each oligonucleotide passes through a pore of the sequencing array. Based on the location of the pore through which the oligonucleotide passes, the protein's location in the tissue section can be determined.

### Example 4. Spatial Gene Expression Analysis of a Biological Sample Using Stimuli for Controlled Release of an Analyte

A tissue section is placed on a glass slide, and the section is prepared for spatial analysis, for example as described in Examples 1 and 2.

If an analyte of interest is a nucleic acid (e.g., mRNA), the steps described in Example 1 (for direct nucleic acid analysis) or in Example 2 (using templated ligation) are used. If an analyte of interest is a protein, then the steps described in Example 3 are used.

In each of the scenarios, the glass slide (e.g., the first substrate) is then aligned with a sequencing array on a second substrate so that some or all of the tissue section aligns with at least some or all of the sequencing array.

A mask can be applied to the first substrate or the second substrate. The mask protects the analyte or intermediate agent from any stimuli that is applied to the substrates to release the analyte or intermediate agent from the tissue section. After (optionally) masking an area of the tissue section, a stimulus such as light (e.g., ultraviolet light) is applied to the tissue section. This stimulus releases the analyte or intermediate agent, allowing it to migrate to the sequencing array on the second substrate. The sequencing array on the second substrate can have hundreds or thousands (e.g., 5,000; 10,000; 15,000) of pores. After sandwiching the glass slide with the second substrate, nucleic acids (e.g., mRNA molecules) migrate from the tissue section to the sequencing array, where each analyte passes through a pore of the sequencing array, in order to sequence the analyte. Based on the location of the pore through which the analyte passes, the analyte's location in the tissue section can be determined. After determining the sequence of the analyte, the mask can be removed (partly or wholly), and the stimulus can be applied to determine the location of the analyte at different location (e.g., a location that was initially masked).

### Example 5. Methods for Sequencing cDNA Using Multiple Substrates

A tissue section is placed on a glass slide, and the section is prepared for spatial analysis, for example as described in Example 1. After, a buffer comprising reverse transcriptase is added to the tissue section, and reverse transcription is performed in situ, generating a plurality of cDNA molecules. The substrate having the tissue section and RT products (e.g., cDNA) is then aligned with a sequencing array on a second substrate so that some or all of the tissue sample aligns with at least some or all of the sequencing array. After aligning the glass slide with the second substrate, a media comprising an RNAse enzyme contacts the tissue sample in order to release the cDNA molecules. The cDNA molecules then migrate from the tissue section to the sequencing array, where each cDNA molecule passes through a pore of the sequencing array, in order to sequence the cDNA molecules. Based on the location of the pore through which the cDNA molecule passes, the analyte's location in the tissue section can be determined.

1. A method for determining location of an analyte or an intermediate agent thereof in a biological sample, the method comprising:
(a) aligning a first substrate comprising the biological sample with a sequencing array on a second substrate, wherein the sequencing array comprises a plurality of pores, such that at least a portion of the biological sample is aligned with at least a portion of the sequencing array on the second substrate;
(b) migrating the analyte or the intermediate agent thereof from the biological sample to the sequencing array; and
(c) passing the analyte or the intermediate agent thereof through a pore of the plurality of pores of the sequencing array, thereby sequencing the analyte or the intermediate agent thereof, and using the sequence of the analyte or the intermediate agent thereof and the location of the pore through which the analyte or the intermediate agent thereof passed to determine the location of the analyte or the intermediate agent thereof in the biological sample.

2. The method of claim 1, wherein the analyte or an intermediate agent thereof is in a region of interest in a biological sample, and wherein the method comprises:
aligning a first substrate comprising the biological sample with the sequencing array such that at least a portion of the biological sample is aligned with at least a portion of the sequencing array on the second substrate; and
releasing the analyte or the intermediate agent thereof from the region of interest in the biological sample

3-4. (canceled)

5. The method of claim 2, wherein the releasing step comprises exposing at least two regions of interest in the biological sample to light, optionally wherein the light exposed to at least two regions of interest is ultraviolet light or infrared light.

6. The method of any claim 5, wherein the releasing step comprises exposing the at least two regions of interest in the biological sample at the same time, optionally wherein analytes or intermediate agents thereof from each region of interest in the at least two regions of interest do not laterally diffuse to other regions of interest.

7-9. (canceled)

10. The method of claim 2, further comprising, prior to the releasing step, applying a mask to the second substrate in a location not aligned with the region of interest.

11. A method for determining location of an analyte or an intermediate agent thereof in a region of interest in a biological sample, the method comprising:
(a) providing a sequencing array on a second substrate, wherein the sequencing array comprises a plurality of pores, optionally aligning a first substrate comprising the biological sample with the sequencing array such that at least a portion of the biological sample is aligned with at least a portion of the sequencing array on the second substrate;
(b) applying a mask to the first substrate in a location not aligned with the region of interest;
(c) releasing the analyte or the intermediate agent thereof from the region of interest in the biological sample;
(d) migrating the analyte or the intermediate agent thereof from the region of interest in the biological sample to a pore of the sequencing array; and
(e) passing the analyte or the intermediate agent thereof through the pore of the sequencing array, thereby sequencing the analyte or the intermediate agent thereof, and using the sequence of the analyte or the intermediate agent thereof and the location of the region of interest in the biological sample to determine expression and location of the analyte or the intermediate agent thereof in the region of interest in the biological sample.

12. The method of claim 11, wherein light is used to release the analyte or the intermediate agent thereof from the region of interest in the biological sample.

13-15. (canceled)

16. The method of claim 11, further comprising, after step (d):
selecting a second region of interest in the biological sample;
repeating steps (b) through (d) to determine a location of a second analyte or a second intermediate agent thereof in the second region of interest in the biological sample; and
sequencing the analyte or the intermediate agent thereof in the biological sample at a first time point followed by sequencing the location of the second analyte or the second intermediate agent thereof in the second region of interest in the biological sample at a second time point, wherein the second time point is after the first time point.

17-18. (canceled)

19. The method of claim 1, wherein the first substrate and/or the second substrate comprises a conductive layer.

20-26. (canceled)

27. The method of any one of claim 1, wherein the analyte or intermediate agent thereof is a nucleic acid, wherein each type of nucleotide in the analyte or the intermediate agent thereof has an electronic signature when passing through the pore of the sequencing array which is distinguishable from the electronic signature of each other type of nucleotide in the analyte or the intermediate agent thereof.

28-42. (canceled)

43. The method of claim 1, wherein the analyte comprises DNA or RNA.

44-48. (canceled)

49. The method of claim 1, further comprising:
hybridizing a first probe and a second probe to the analyte in the biological sample, wherein the analyte is a nucleic acid, wherein the first probe and the second probe each comprise a sequence that is substantially complementary to sequences of the analyte; and
coupling the first probe and the second probe, thereby generating a connected probe hybridized to the analyte in the biological sample.

50-53. (canceled)

54. The method of claim 49, wherein the first probe and the second probe hybridize to sequences in the analyte that are at least 1, 2, 3, 4, 5, 6, 7, 8, 9, 10, or more nucleotides apart.

55-60. (canceled)

61. The method of claim 1, wherein the analyte is a protein.

62. The method of claim 61, further comprising contacting the biological sample with a plurality of analyte capture agents, wherein an analyte capture agent of the plurality of analyte capture agents comprises an analyte binding moiety and a capture agent barcode domain, wherein the capture agent barcode domain comprises an analyte binding moiety barcode that uniquely identifies the analyte binding moiety and/or the protein, and wherein upon the contacting, the analyte binding moiety binds to the protein in the biological sample.

63-67. (canceled)

68. The method of claim 1, wherein the biological sample is a tissue sample.

69-71. (canceled)

72. A system comprising:
(a) a first substrate comprising a biological sample;
(b) a second substrate comprising a sequencing array comprising a plurality of pores; and
(c) optionally a flow cell between the first substrate and the second substrate.

73. The system of claim 72, further comprising:
a light source, optionally wherein the light source is for releasing an analyte from the biological sample; and
a mask to inhibit release of analytes in regions that are not of interest in the biological sample.

74. (canceled)

75. The system of claim 72, wherein the system further comprises one or more of a reverse transcriptase, a polymerase, a plurality of dNTPs, and one or more permeabilization reagents.

76-86. (canceled)

87. A kit comprising:
(a) a first substrate comprising a biological sample;
(b) a second substrate comprising a sequencing array comprising a plurality of pores; and
(c) instructions for performing the method of claim 1.

88-106. (canceled)
